(19) United States
(12) Patent Application Publication
(10) Pub. No.: US 2005/0222531 A1
(43) Pub. Date: Oct. 6, 2005

(54) Ankle braces

(76) Inventor: Timothy Ian Moore, Hythe (GB)

Correspondence Address: JOHN V STEWART, 1308 HENRY BALCH DRIVE, ORLANDO, FL, 32810, US

(21) Appl. No.: 10/518,975

(22) PCT Filed: Jun. 20, 2003
(86) PCT No.: PCT/GB03/02666

§ 371 Date: Dec. 21, 2004

(30) Foreign Application Priority Data
Jun. 22, 2002 (GB) 0214472.3

(51) Int. Cl. A61F 5/00

(52) U.S. Cl. 602/27; 602/3

(57) ABSTRACT

A shell (14) for an ankle brace is semi-rigid and has an inner surface shaped to conform to a side surface of a person's leg (18) between an upper extent (62) above the person's ankle bone (30) and a lower extent (50) below the person's ankle bone but above the base of the heel, and between a forward extent (42, 46) towards the front of the person's leg and a rearward extent (44, 48) towards the rear of the person's leg. The shell has a portion (28) shaped to register the shell with respect to the person's ankle bone (30). The shell is bifurcated by a slot (60) extending from the upper extent towards the registration portion. By registering the shell with respect to the ankle bone, a better fit can be achieved. The bifurcation of the shell facilitates flexing of the shell so that it can adapt to the curvature of the surface of the leg, so as to provide a more comfortable fit of the shell and a more even pressure distribution than if the shell were not bifurcated.

This invention relates to orthopaedic devices, in particular to ankle braces and to shells used in ankle braces, and to methods of bracing the ankle.

After injury to an ankle, such as a fracture or severe ankle sprain, it may be necessary to completely immobilize the ankle through the use of a moulded plaster or resin cast. However, once the injury has been stabilized, recovery may be hastened by removing the cast and using, in its place, a removable, functional walking brace. The brace stabilizes the ankle against inversion, eversion and anterior subluxation, whilst still permitting normal dorsiflexion and plantarflexion movement. This action accelerates the natural healing process and shortens the time required to rehabilitate the injury. Such braces may also be used to prevent an initial ankle injury or reoccurrence of an injury.

The main ankle ligaments are the anterior talofibular, the calcaneofibula, the posterior talofibular and the deltoid. It is estimated that 85% of ankle sprains involve both the calcaneofibular and anterior talofibular ligaments together and 70% of sprains due to inversion involve the anterior talofibular ligament alone. The correct assessment and treatment of a patient is often delayed due to the swelling caused by the trauma. No treatment, or else incorrect treatment, is frequently given because no proper diagnosis can be made.

A number of different designs of ankle brace are known. Most of then have a pair of rigid, or semi-rigid, side supports that are strapped to the sides of the lower leg and ankle. Some side supports may have an integral joining piece, or strap, that extends under the heel. Furthermore, no ankle brace mentioned below provides support for all of the ligaments; there are deficiencies in these existing designs that the present invention is aimed at alleviating.

A summary of the state of the relevant art is contained in the introduction of patent document WO-A-02/051343, to which reference is directed, the content of which is incorporated herein by reference, and which is hereinafter referred to as “the earlier application.” Also, patent document U.S. Pat. No. 4,719,926 describes an ankle brace comprising a bracket that extends under the heel and up the side of the foot to a level below the ankle bone, a narrow-limbed U-shaped member that is pivotally and slidably attached to the bracket, and strapping for securing the U-shaped member to the side of the leg so that it extends down the leg, around and underneath the ankle bone and back up the leg. This brace attempts to provide an artificial hinge between the leg and the foot having its hinge axis coaxial with the axis of the ankle. However, (a) it provides little support for the ligaments of the ankle, (b) it registers itself with the base of the heel and is not adjustable with regard to the height of the ankle axis above the base of the heel, (c) it is likely to be uncomfortable to wear particularly in view of the narrowness of the limbs of the U-shaped member, and (d) the sliding action of the U-shaped member with respect to the bracket is questionable. Furthermore, patent document U.S. Pat. No. 4,495,942 describes an ankle brace comprising a U-shaped felt pad intended to be placed around the lateral ankle bone and an elastic strap that is connected to the felt pad and intended to be wound around the ankle, foot and pad to apply pressure to the pad and ankle and maintain the ankle and foot in a position of stability. This brace therefore acts similarly to conventional ankle strapping or taping, but with additional padding to the front of, rear of and below the ankle bone.

The present invention, or specific embodiments of it, is concerned with providing an effective and comfortable ankle brace or ankle brace shell.

In accordance with a first aspect of the present invention, there is provided a shell for an ankle brace, the shell being semi-rigid and having an inner surface shaped to conform to a side surface of a person's leg between an upper extent above the person's ankle bone and a lower extent below the person's ankle bone but above the base of the heel, and between a forward extent towards the front of the person's leg and a rearward extent towards the rear of the person's leg, the shell having a portion shaped to register the shell with respect to the person's ankle bone, and the shell being bifurcated by a slot extending from the upper extent towards the registration portion.

Unlike the ankle brace of U.S. Pat. No. 4,719,926, the shell of this aspect of the invention provides support for the ankle without actually acting as an artificial ankle joint. The shell is shaped so that the shell itself registers itself with the ankle bone, by contrast to the brace of U.S. Pat. No. 4,719,926 that is not registered with respect to the ankle and that has a separate bracket to register the U-shaped member with the base of the heel. By registering the shell with respect to the ankle bone, a better fit can be achieved. The bifurcation of the shell facilitates flexing of the shell so that it can adapt to the curvature of the surface of the leg, as viewed in the axial direction of the leg, so as to provide a more comfortable fit of the shell and a more even pressure distribution than if the shell were not bifurcated. Increased comfort encourages the patient to wear the ankle brace.

The width of the slot above the registration portion is preferably at least 5 mm, so that there is little risk of skin being pinched in the slot and preferably at most 30 mm so as not to lose too much rigidity in the shell. Preferably, the width of each portion of the shell to either side of the slot is greater than the width of the slot.

Preferably, the registration portion is provided by a hole for snugly receiving the protruding part of the ankle bone. The hole may be dimensioned similarly to that described in the earlier application. In this case, the slot preferably opens into the hole. However, the slot may stop short of the hole.

However, alternatively, the registration portion may be provided by a recess in the inner surface of the shell for receiving the protruding part of the ankle bone. In this case, the slot may stop short of, extend as far as, or extend at least partly into, the recess.

Preferably, the registration portion has a non-circular outline. For example, it may be generally elliptical as described in the earlier application.

Preferably, the shell has a generally uniform thickness.

The shell may be provided in combination with means for adjustably strapping the shell to the person's leg. The shell is preferably devoid of any feature extending beneath the person's heel for non-adjustably registering the shell vertically with respect to the person's leg.

Preferably, the forward extent of the shell has a projecting region for covering the person's anterior talofibular ligament, as described in more detail in the earlier application. In this case, the projecting region preferably projects by a maximum distance of about 60 mm±10 mm from the tip of the person's ankle bone.

In accordance with a second aspect of the invention, there is provided an ankle brace comprising a medial shell according to the first aspect of the invention for the medial side surface of the person's leg, a lateral shell according to the first aspect of the invention for the lateral side surface of the person's leg, and means for adjustably strapping the shells to the person's leg.

Preferably, the strapping means includes an adjustable strap for extending under the person's heel between the lower extents of the shells.

In accordance with a third aspect of the invention, there is provided an ankle brace comprising a sock having at least one shell according to the first aspect of the invention fitted to the sock.

In accordance with a fourth aspect of the invention, there is provided an ankle brace comprising a shoe or boot having at least one shell according to the first aspect of the invention fitted into the shoe or boot.

In accordance with a fifth aspect of the invention, there is provided a method of preventing injury to a person's ankle, or a method of treatment of an injury to a person's ankle, comprising fitting a shell or an ankle brace according to the earlier aspects of the invention to the person's leg.

A specific embodiment of the present invention will now be described, purely by way of example, with reference to the accompanying drawings, in which:

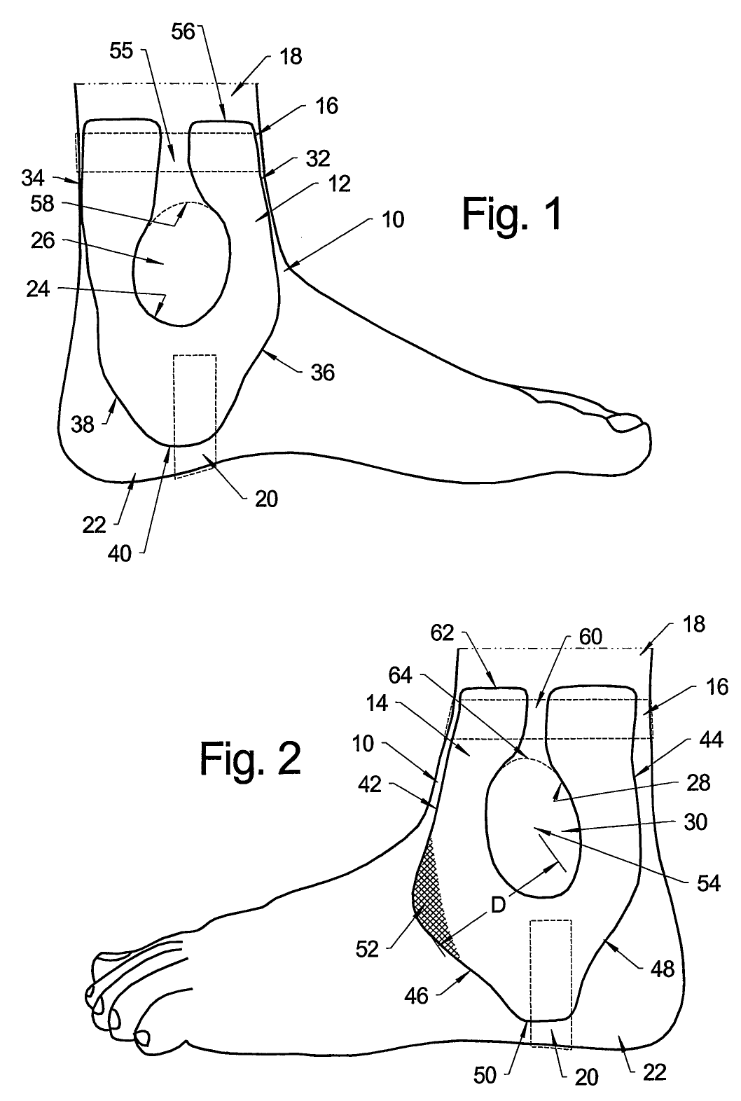

FIG. 1 is a view of the medial (or inner) side of the person's lower leg, ankle and foot with the ankle brace fitted thereto;

FIG. 2 is a view of the lateral (or outer) side of a person's lower leg, ankle and foot with an ankle brace fitted thereto;

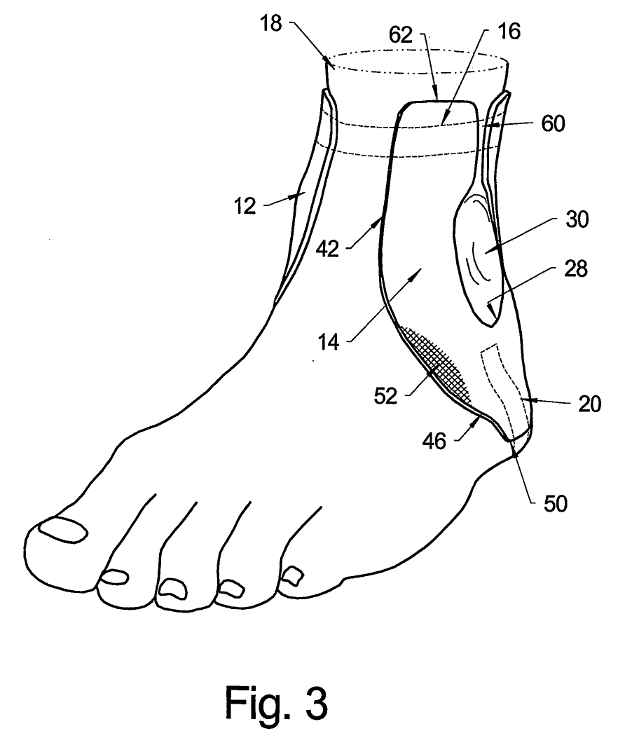

FIG. 3 is a front view looking slightly downwards and from the lateral side of the ankle brace fitted to the person's foot;

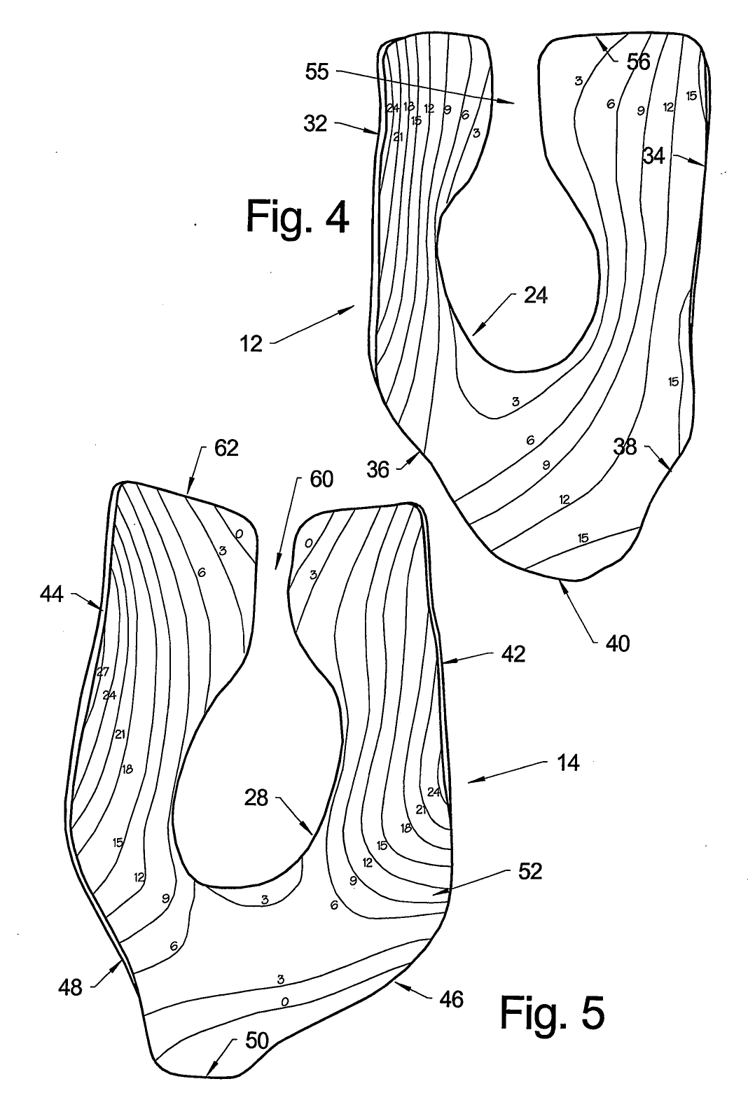

FIG. 4 is a view of the inner face of a medial shell of the ankle brace seen in FIG. 1, marked with contour lines; and

FIG. 5 is a view of the inner face of a lateral shell of the ankle brace seen in FIG. 2, marked with contour lines.

Referring to the drawings, the ankle brace 10 comprises a medial (inner) shell 12, a lateral (outer) shell, 14, and strapping (shown schematically in FIGS. 1 and 2 by dashed lines) for attaching the brace around a person's ankle. The strapping comprises one or more straps 16, that extends around the person's leg 18, and a lower strap 20 that extends under the person's heel 22. The effective length of each strap 16,20 is independently adjustable, for example using Velcro®.

The shells 12,14 are formed of semi-rigid material, such as vacuum-formed plastics, injection-moulded plastics, glass reinforced plastics, graphite/glass reinforced plastics or metal. Each shell has a generally uniform thickness of, for example, 3 mm to 4 mm. The medial shell 12 has a generally elliptical hole 24 that receives the protruding part 26 of the person's body covering the medial end of the anklebone. Similarly, the lateral shell 14 has a generally elliptical hole 28 that receives the protruding part 30 of the person's body covering the lateral end of the anklebone.

The inner surface of the medial shell 12 is shaped to conform closely to the medial side surface of the person's leg 18 that it covers. FIG. 4 shows contour lines for the inner surface of the medial shell 12 marked in millimetres relative to an arbitrary datum plane. Accordingly, when the medial shell 12 is applied under pressure to the medial side surface of the person's leg 18, the hole 24 registers the medial shell 12 with respect to the person's anklebone, and substantially the whole of the inner surface of the medial shell 12 contacts the person with a generally uniform pressure distribution, but without any substantial pressure being applied to the protruding part 26 of the person's body covering the medial end of their anklebone.

Similarly, the inner surface of the lateral shell 14 is shaped to conform closely to the lateral side surface of the person's leg 18 that it covers. FIG. 5 shows contour lines for the inner surface of the lateral shell 14 marked in millimetres relative to an arbitrary datum plane. Accordingly, when the lateral shell 14 is applied under pressure to the lateral surface of the person's leg 18, the hole 28 registers the lateral shell 14 with respect to the person's anklebone, and substantially the whole of the inner surface of the lateral shell 14 contacts the person with a generally uniform pressure distribution, but without any substantial pressure being applied to the protruding part 30 of the person's body covering the lateral end of their anklebone.

Over generally the upper half of the medial shell 12, the upper front and rear edges 32,34 of the medial shell 12 are generally parallel. Over generally the lower half of the medial shell 12, the lower front and rear edges 36,38 of the medial shell 12 converge towards the lowermost extent 40 of the medial shell 12. By contrast, over generally the upper half of the lateral shell 14, the upper front and rear edges 42,44 of the lateral shell 14 diverge slightly, and then over generally the lower half of the lateral shell 14, the lower front and rear edges 46,48 of the lateral shell 14 converge towards the lowermost extent 50 of the lateral shell 14. This produces a projecting portion 52 part way along the front edge 42,46 of the lateral shell 14 that covers and provides support for the person's anterior talofibular ligament. The projecting portion 52 projects a maximum distance D of about 60 mm±10 mm from the lateral tip 54 of the person's ankle bone.

As described above, the projecting portion 52 is integral with and of the same material as the remainder of the lateral shell 14. If desired, the projecting portion 52 may be thinned, or thickened, so that it is more, or less, flexible. Furthermore, the projecting portion 52 may be formed of a different material that is bonded to the remainder of the lateral shell 14 and that is more, or less, flexible than the remainder of the lateral shell 14. Thus the degree of support for the anterior talofibular ligament may be controlled. In cases where the projecting portion 52 on the lateral shell 14 is not required, or is only required in the initial stages of treatment, the projecting portion 52 may be cut-off from the remainder of the lateral shell 14. Alternatively, the projecting portion 52 may be removably attachable to the reminder of the lateral shell 14, and different sizes, shapes and/or thicknesses of the projecting portion 52 may be provided so that a projecting portion 52 with the required properties can be selected and fitted to the lateral shell 14, or so that the lateral shell 14 may be used without a projecting portion 52. Similar modifications and options may be provided for the forwardly-bulging forward extent of the medial shell 12.

The medial shell 12 is formed with a slot 55 that extends generally vertically between the upper edge 56 and the hole 24 of the medial shell 12 so that, overall, the medial shell 12 is a generally wide-limbed U-shape. The width of the slot 55 is preferably at least 5 mm so that the person's skin does not become pinched in the slot 55 as the medial shell 12 flexes, and is preferably at most 30 mm so as not to lose too much rigidity in the limbs of the medial shell 12 to either side of the slot 55. More preferably, the width of the slot 55 is about 10, 15, 20 or 25 mm. In particular, the width of the slot 55 is less that the width of the hole 24, so that the hole 24 snugly encircles the protruding part 26 of the person's body covering the medial end of their anklebone, except over a minor portion indicated by the dashed line 58 in FIG. 1. Accordingly, despite the provision of the slot 55, the hole 24 can register the medial shell 12, both horizontally and vertically, with respect to the protruding part 26 of the person's body covering the medial end of their anklebone.

Similarly, the lateral shell 14 is formed with a slot 60 that extends generally vertically between the upper edge 62 and the hole 28 of the lateral shell 14 so that, overall, the lateral shell 14 is a generally wide-limbed U-shape. Preferably, the width of the slot 60 is at least 5 mm so that the person's skin does not become pinched in the slot 60 as the lateral shell 14 flexes, and is at most 30 mm so as not to lose too much rigidity in the limbs of the lateral shell 14 to either side of the slot 60. More preferably, the width of the slot 60 is about 10, 15, 20 or 25 mm. In particular, the width of the slot 60 is less that the width of the hole 28, so that the hole 28 snugly encircles the protruding part 30 of the person's body covering the lateral end of their anklebone, except over a minor portion indicated by the dashed line 64 in FIG. 2. Accordingly, despite the provision of the slot 60, the hole 28 can register the lateral shell 14, both horizontally and vertically, with respect to the protruding part 30 of the person's body covering the lateral end of their anklebone.

When the brace 10 is initially fitted to the person, the medial and lateral shells 12,14 are placed in position against the medial and lateral sides, respectively, of the person's leg 18 and with the ankle portions 26,30 registering with the holes 24,28. Due to the general shape, size and configuration of the shells 12,14, they readily assume their correct positions. The straps 16,20 are then fastened and individually adjusted in length so that the shells 12,14 apply light pressure to the ankle area when the ankle is in a relaxed state. The slots 55,60 facilitate flexing of the shells 12,14 so that they adapt to the curvature of the surface of the leg 18, as viewed in the axial direction of the leg 18, so as to provide a more comfortable fit of the shells 12,14 and a more even pressure distribution than if the slots 55,60 were not provided.

A sock may be worn underneath the brace 10. Due to the thinness of the shells 12,14 and the lack of any large domed protrusions at the ends of the anklebone, it is likely that a conventional shoe or boot may be worn over the sock and anklebrace. If the brace 10 is initially applied when the ankle is swollen, then as the swelling subsides the straps 16,20 can be progressively shortened to maintain a good fit of the brace 10 with the parts 26,30 of the body covering the ends of the anklebone progressively protruding further into the holes 24,28.

It will be appreciated that many modifications and developments may be made to the embodiment of the invention. For example, the sizes of the shells may be scaled for different sizes of ankle. Also, the shells 12,14 may be used singly, rather than as a pair. Also, the inner surfaces of the shell 12,14 may be provided with a layer of padding material, but preferably the padding is relatively firm and relatively thin so as neither to reduce unduly the stability of the shell nor to increase unduly the bulkiness of the brace. The shells 12,14 may be formed with additional perforations through their thickness to allow the user's leg/ankle to breathe and thus reduce problems of moisture retention, and any padding may likewise be perforated or porous. Some features of the shape and size of the shells 12,14 may be modified. For example, the height of the shells 12,14 above the holes 30,34 may be varied considerably. Also, the shape and extent of the shells 12,14 below the holes 30,34 may be varied. One or both shells 12,14 may be permanently or detachably fitted to a sock or boot.

Although it is preferred that the holes 24,28 are provided in the shells 12,14, the shells 12,14 may be continuous over the protruding parts 26,30 of the person's body covering the ends of their anklebone, but recessed or dished so as to accommodate the protruding parts 26,30. Also, although it is preferred that the slots 55,60 extend downwardly from the upper edges 56,62 of the shells 12,14 as far as the holes 24,28 (or recesses), they may extend only part-way, or in the case where recesses rather than holes 24,28 are provided the slots 55,60 may extend downwardly part-way into the recesses.

It should be noted that the embodiment of the invention has been described above purely by way of example and that many modifications and developments may be made thereto within the scope of the present invention.

1. A shell (12;14) for an ankle brace (10), the shell being semi-rigid and having an inner surface shaped to conform to a side surface of a person's leg (18) between an upper extent (56;62) above the person's ankle bone (26;30) and a lower extent (40;50) below the person's ankle bone but above the base of the heel (22), and between a forward extent (32,36;42,46) towards the front of the person's leg and a rearward extent (34,38;44,48) towards the rear of the person's leg, the shell having a portion (24;28) shaped to register the shell with respect to the person's ankle bone, and the shell being bifurcated by a slot (55;60) extending from the upper extent towards the registration portion.

2. A shell as claimed in claim 1, wherein the width of the slot above the registration portion is at least 5 mm.

3. A shell as claimed in claim 1, wherein the width of the slot above the registration portion is at most 30 mm.

4. A shell as claimed in claim 1, wherein the registration portion is provided by a hole (24;28) for snugly receiving the protruding part of the ankle bone.

5. A shell as claimed in claim 4, wherein the slot opens into the hole.

6. A shell as claimed in claim 4, wherein the slot stops short of the hole.

7. A shell as claimed in claim 1, wherein the registration portion is provided by a recess in the inner surface of the shell for receiving the protruding part of the ankle bone.

8. (canceled)

9. A shell as claimed in claim 7, wherein the slot extends as far as the recess.

10. A shell as claimed in claim 7, wherein the slot extends at least partly into the recess.

11. A shell as claimed in claim 1, wherein the registration portion has a non-circular outline.

12. A shell as claimed in claim 1, wherein the shell has a generally uniform thickness.

13. A shell as claimed in claim 1, in combination with means (16,20) for adjustably strapping the shell to the person's leg.

14. A shell as claimed in claim 1, wherein the shell is devoid of any feature extending beneath the person's heel for non-adjustably registering the shell vertically with respect to the person's leg.

15. A shell as claimed in claim 1, wherein the forward extent of the shell has a projecting region (52) for covering the person's anterior talofibular ligament.

16. A shell as claimed in claim 15, wherein the projecting region projects by a maximum distance (D) of about 60 mm±10 mm from the tip (54) of the person's ankle bone (30).

17. An ankle brace (10) comprising a medial shell (12), the medial shell being semi-rigid and having an inner surface shaped to conform to the medial side surface of a person's leg between an upper extent (56) above the person's ankle bone (26) and a lower extent (40) below the person's ankle bone but above the base of the heel (22), and between a forward extent (32,36) towards the front of the person's leg and a rearward extent (34,38) towards the rear of the person's leg, a lateral shell (14), the lateral shell being semi-rigid and having an inner surface shaped to conform to the lateral side surface of the person's leg between an upper extent (62) above the person's ankle bone (30) and a lower extent (50) below the person's ankle bone but above the base of the heel (22), and between a forward extent (42,46) towards the front of the person's leg and a rearward extent (44,48) towards the rear of the person's leg, and means (16,20) for adjustably strapping the shells to the person's leg, each shell having a respective portion (24;28) shaped to register that shell with respect to the person's ankle bone, and each shell being bifurcated by a respective slot (55,60) extending from the respective upper extent towards the respective registration portion.

18. An ankle brace as claimed in claim 17, wherein the strapping means includes an adjustable strap (20) for extending under the person's heel (22) between the lower extents of the shells.

19. A shell as claimed in claim 1 in combination with a sock, shoe or boot, the shell being fitted to the sock, shoe or boot.

20. (canceled)

21. A shell (12;14) for an ankle brace (10), the shell being semi-rigid and having an inner surface shaped to conform to a side surface of a person's leg (18) between an upper extent (56;62) above the person's ankle bone (26;30) and a lower extent (40;50) below the person's ankle bone but above the base of the heel (22), and between a forward extent (32,36;42,46) towards the front of the person's leg and a rearward extent (34,38;44,48) towards the rear of the person's leg, the shell having a portion (24;28) shaped to register the shell with respect to the person's ankle bone, and the forward extent of the shell having a projecting region (52) for covering the person's anterior talofibular ligament.

22. A shell as claimed in claim 21, wherein the projecting region projects by a maximum distance (D) of about 60 mm±10 mm from the tip (54) of the person's ankle bone (30).
